(19) United States
(12) Patent Application Publication
(10) Pub. No.: US 2014/0277509 A1
(43) Pub. Date: Sep. 18, 2014

(54) EXPANDABLE VERTEBRAL BODY REPLACEMENT AND METHODS

(71) Applicants: James C. Robinson, Atlanta, GA (US); John E. Pendleton, Atlanta, GA (US)

(72) Inventors: James C. Robinson, Atlanta, GA (US); John E. Pendleton, Atlanta, GA (US)

(73) Assignee: Spectrum Spine IP Holdings, LLC, Atlanta, GA (US)

(21) Appl. No.: 14/216,513

(22) Filed: Mar. 17, 2014

Related U.S. Application Data
(60) Provisional application No. 61/802,360, filed on Mar. 15, 2013.

(51) Int. Cl. A61F 2/44

(52) U.S. Cl. CPC A61F 2/447
USPC 623/17.16

(57) ABSTRACT

An expandable vertebral body replacement is presented. The device has an inner and outer wall longitudinally moveable on one-another which locks in place using a ratchet mechanism. This can be locked or fortified by several described options. Also presented is a method for expanding said device embodiments.

### CONTINUITY

This application claims priority to and the benefit of U.S. Provisional Patent Application No. 61/802,360, filed on Mar. 15, 2013, the contents of which are incorporated herein in their entirety.

### FIELD OF THE INVENTION

This invention relates generally to spinal surgery, and more particularly to devices and methods of stabilization of the spine following removal of a vertebral body, and replacing it with an expandable construct to stabilize the adjacent bones and provide a corridor between them for fusion purposes.

### BACKGROUND OF THE INVENTION

Damage to a vertebral bone, often due to a traumatic fracture or due to invasion of the bone by tumor, at times requires removal of the vertebral body. This operation is known as a corpectomy. Following corpectomy, the resultant gap is generally filled in by a weight bearing support know as a Vertebral Body Replacement (VBR). This helps to restore and maintain the proper spacing between the adjacent bones, and often provides an area for placing graft material to span the adjacent bones in order to allow a fusion to take place.

VBRs may be sized to fit the gap, or be expandable over a range. The expandable VBRs currently available have significant limitations. These include the inability to adequately pack graft material in the channel within them post-expansion, a lack of a satisfactory match of the top and bottom of the implant to the adjacent bone surfaces, and expansion mechanisms that are complex, or take an inordinate amount of time to actuate. A need remains for an implant and surgical technique that will overcome these shortfalls.

### SUMMARY

Presented herein is an expandable VBR device for use in spinal surgery following a vertebral corpectomy in the cervical, thoracic, or lumbar spine. The VBR device comprises a first housing and a second housing. The first and second housings can move in relation to one another in the longitudinal direction to increase or reduce the height of the VBR device. The VBR device can be expanded within a range from the first, unexpanded position, to a second fully expanded position or substantially any position therebetween.

A method of placing an expandable VBR into a corpectomy defect, and expanding the height of the device using a VBR expansion tool is also presented. The method comprises, accessing the desired motion segment, removing the desired vertebral body, positioning the expandable VBR device in place of the removed vertebral body, expanding the expandable VBR device, and fixing the expandable VBR device in the expanded position. The method can also comprise fixing the lordotic angle of the upper and/or lower bone contact members. In another aspect, the method also comprises packing the graft cavity with bone growth promoting materials.

Related methods are also provided. Other apparatuses, methods, systems, features, and advantages of the expandable VBR device and the method of its use will be or become apparent to one with skill in the art upon examination of the following figures and detailed description. It is intended that all such additional apparatuses, methods, systems, features, and advantages be included within this description, be within the scope of the expandable VBR device and the method of its use, and be protected by the accompanying claims.

### DESCRIPTION OF THE FIGURES

The accompanying drawings, which are incorporated in and constitute a part of this specification, illustrate certain aspects of the instant invention and together with the description, serve to explain, without limitation, the principles of the invention. Like reference characters used therein indicate like parts throughout the several drawings.

FIG. 1 is a partially exploded front perspective view of one aspect of a VBR device;

FIG. 2 is a partially exploded perspective view of the VBR device of FIG. 1;

FIG. 3 is a partially exploded side view of the VBR device of FIG. 1;

FIG. 4 is a partially exploded front view of the VBR device of FIG. 1;

FIG. 5 is a top plan view of the VBR device of FIG. 1;

FIG. 6 is a front view of another aspect of a VBR device in the unexpanded position;

FIG. 7 is a perspective view of the VBR device of FIG. 6 in an expanded position;

FIG. 8 is a top plan view of the VBR device of FIG. 6;

FIG. 9 is a cut away front view of the VBR device of FIG. 6, showing a ratchet mechanism for restraining the VBR from moving into the retracted position from the expanded position;

FIG. 10 is a partial perspective view of an interior wall of an inner sleeve of the VBR device of FIG. 6, showing the relationship of a tooth positioned on the interior wall of the outer sleeve with a recess defined in the exterior wall of the inner sleeve;

FIG. 11 is a cutaway view of the engagement of the inner and outer sleeve of the VBR device of FIG. 10;

FIG. 12 is a perspective view of the VBR device of FIG. 6, showing the VBR device positioned between two vertebrae;

FIG. 13 is a perspective view of the VBR device of FIG. 6, showing the VBR device with a VBR expansion tool;

FIG. 14 is a front view of one aspect of a VBR device showing an inner housing and an outer housing;

FIG. 15 is a perspective view of the VBR device of FIG. 14;

FIG. 16 is a cut away front view of the VBR device of FIG. 14, showing gear engaged with a toothed inner surface of the inner housing;

FIG. 17 is a top plan view of a retention member; and

FIG. 18 is a perspective view of the retention member of FIG. 17.

### DESCRIPTION OF THE INVENTION

The present invention can be understood more readily by reference to the following detailed description, examples, and claims, and their previous and following description. Before the present system, devices, and/or methods are disclosed and described, it is to be understood that this invention is not limited to the specific systems, devices, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. Those skilled in the relevant art will recognize that many changes can be made to the aspects described, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used herein, the singular forms “a,” “an” and “the” include plural referents unless the context clearly dictates otherwise. Thus, for example, reference to a “spike” includes aspects having two or more spikes unless the context clearly indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

A description of an element of the aspect of the invention that refers to opposed portions of the invention such as upper and lower, left and right, inner and outer also includes the inverse relationship where practical. For example, reference to an upper housing nested within a lower housing includes a lower housing nested within an upper housing where such a relationship does not materially change the aspect of the invention.

As used herein, the terms “optional” or “optionally” mean that the subsequently described event or circumstance may or may not occur, and that the description includes instances where said event or circumstance occurs and instances where it does not.

Terms used herein, such as “exemplary” or “exemplified,” are not meant to show preference, but rather to explain that the aspect discussed thereafter is merely one example of the aspect presented.

Additionally, as used herein, relative terms, such as “substantially”, “generally”, “approximately”, and the like, are utilized herein to represent an inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

In one aspect, presented herein is an expandable VBR device 10 for use in spinal surgery following a vertebral corpectomy in the cervical, thoracic, or lumbar spine. The VBR device comprises an inner housing 100 and an outer housing 200. The inner housing 100 and outer housing 200 can move in relation to one another in the longitudinal direction to increase or reduce the height of the VBR device 10. In one exemplified aspect, the outer housing defines in interior cavity 210 within which the inner housing can nest in the unexpanded position. In another aspect, the inner housing 100 and outer housing 200, together, define a graft cavity 215 for the placement of bone graft material, to include, but not limited to, allograft, bone substitute, or other biocompatible bone growth promoting materials.

The VBR device can be expanded within a range from the first, unexpanded position, to a second fully expanded position or substantially any position therebetween. In an exemplified aspect, an interior surface 220 of the outer housing 200 comprises a toothed surface 225. In one aspect, the exterior surface 110 of the inner housing 100 comprises a toothed surface 125, as well. Each tooth can comprise a cam 130, 230 surface and a flat surface 135, 235. In one aspect, the toothed surfaces are complimentarily opposite, meaning that in the expanding direction, the cam surface of a tooth on the outer housing mates with the cam surface of a tooth on the inner housing and, in the retracting direction, the flat surface of a tooth on the outer housing engages the flat surface of a tooth on the inner housing This relationship permits the two housings to move in the expanding direction in a ratcheting manner, but prevents the two housings to move in the retracting direction.

In another exemplified aspect, each tooth 125, 225 can comprise two cam surfaces 130, 230, without a flat surface. Additionally, the VBR device can comprise a retention member 300 configured to engage at least a portion of the toothed surface of the inner housing to prevent longitudinal up and down movement of the outer housing with respect to the inner housing. In one aspect, the retention member can be a u-shaped member sized to horizontally slide around at least at least portions the exterior surface 110 of the inner housing 100.

In still another exemplified aspect, the inner housing may define a window 160 having a toothed edge portion 165. In this aspect, a gear 260 can be positioned on the outer housing 200 configured to engage the toothed edge portion 165 of the window 160 of the inner housing 100. In one aspect, the exterior surface 110 of the inner housing may comprise a toothed surface 125. The VBR device can also comprise a retention member 300 configured to engage the exterior surface of the inner housing to restrict movement of the inner housing with respect to the outer housing. The retention member can be a c-ring configured to at least partially wrap around the inner housing and engage at least one of the teeth on the external surface of the inner housing.

In an exemplified aspect, the VBR device comprises an upper bone contact member 400 and a lower bone contact member 450. The upper bone contact member 400 has an upper bone contact surface 410 configured for contact with a lower portion of a first vertebra. The lower bone contact member 450 has a lower bone contact surface 460 configured for contact with an upper portion of a second vertebra. One or both of the upper and lower bone contact members can be substantially planar. In one aspect, the upper bone contact member can be pivotally connected to a portion of the top portion 240 of the outer housing. The pivot point 415 can, for example, be in substantially the center of the of the upper bone contact member, enable the upper bone contact member to angulate in either the anterior or posterior direction. The function permits the VBR device to adapt to the anatomy of the patient. As can be appreciated, the lower bone contact member 450 can be pivotally connected to a portion of the bottom portion of the inner housing. The pivot point 465 can, for example, be in substantially the center of the of the lower bone contact member, enable the lower bone contact member to angulate in either the anterior or posterior direction. One or both of the upper and lower bone contact members can comprise spikes 600 or protrusions to facilitate engagement with the respective vertebral bone.

In still another aspect, the VBR device comprises an inner and outer sleeve 500, 550, wherein the outer and inner sleeves are configured to move longitudinally with respect to one another from the retracted position to the expanded position. The inner and outer sleeve define an interior cavity 555. In an exemplified aspect, an interior surface 560 of the outer sleeve 550 comprises a toothed surface 565. In one aspect, the exterior surface 510 of the inner sleeve 500 comprises a toothed surface 515, as well. Each tooth can comprise a cam surface 516, 566 and a flat surface 517, 567. In one aspect, the toothed surfaces are complimentarily opposite, meaning that in the expanding direction, the cam surface of a tooth on the outer sleeve mates with the cam surface of a tooth on the inner sleeve and, in the retracting direction, the flat surface of a tooth on the outer sleeve engages the flat surface of a tooth on the inner sleeve This relationship permits the two sleeves to move in the expanding direction in a ratcheting manner, but prevents the two sleeves to move in the retracting direction. As one skilled in the art can appreciate, in lieu of complimentary toothed surfaces, the inner and outer sleeves can comprise one toothed surface and define a complimentary recess 520 on the other surface, where the recess 520 has a flat interior edge 522 that compliments the flat surface 567 of the tooth of the other surface.

In this aspect, the upper bone contact member can be hingedly attached to a top portion 580 of the outer sleeve and the lower bone contact member can be hingedly attached to a lower portion 530 of the inner sleeve. This configuration permits angulation of the bone contact members to compliment the adjacent vertebral bodies. In an exemplified aspect, the inner and outer sleeves nest within one another and define a pillar window 570. In this aspect, the VBR device comprise a pillar 20 configured to fit within the interior cavity 555 to be placed into the interior cavity via the pillar window 570. In an exemplified aspect, a top portion 21 of the pillar is angled and the bottom portion of the pillar 22 is also angled. The top portion 21 of the pillar engages a portion of the upper bone contact member 400 and the angle of the top portion of the pillar places the upper bone contact member to the desired angle for lordosis. Similarly, the bottom portion 22 of the pillar engages a portion of the lower bone contact member 450 and the angle of the lower portion of pillar places the lower bone contact member to the desired angle for lordosis. The pillar, of course, substantially prevents the VBR device from moving from the expanded position to the retracted position.

A method of placing an expandable VBR into a corpectomy defect, and expanding the height of the device using a VBR expansion tool is presented. The method comprises, accessing the desired motion segment, removing the desired vertebral body, positioning the expandable VBR device in place of the removed vertebral body, expanding the expandable VBR device, and fixing the expandable VBR device in the expanded position. The method can also comprise fixing the lordotic angle of the upper and/or lower bone contact members. In another aspect, the method also comprises packing the graft cavity with bone growth promoting materials.

Although several aspects of the invention have been disclosed in the foregoing specification, it is understood by those skilled in the art that many modifications and other aspects of the invention will come to mind to which the invention pertains, having the benefit of the teaching presented in the foregoing description and associated drawings. It is thus understood that the invention is not limited to the specific aspects disclosed hereinabove, and that many modifications and other aspects are intended to be included within the scope of the appended claims. Moreover, although specific terms are employed herein, as well as in the claims that follow, they are used only in a generic and descriptive sense, and not for the purposes of limiting the described invention.

What is claimed is:

1. A vertebral body replacement device comprising:
a first housing defining an interior cavity;
a second housing nesting within at least a portion of the interior cavity of the first housing, the second housing moveable along a longitudinal direction to increase a height of the vertebral body replacement device;
means for preventing contraction of the vertebral body replacement device while permitting expansion of the vertebral body replacement device;
a first bone contact member having a first bone contact surface: and
a second bone contact member having a second bone contact surface.

2. The vertebral body replacement device of claim 1, wherein the second housing and first housing define a graft cavity for the placement of bone graft material.

3. The vertebral body replacement device of claim 1, wherein the first bone contact member is pivotally connected to a portion of a top portion of the first housing enabling the first bone contact member to angulate in either an anterior or posterior direction.

4. The vertebral body replacement device of claim 1, wherein the second bone contact member is pivotally connected to a portion of a lower portion of the second housing enabling the second bone contact member to angulate in either an anterior or posterior direction.

5. The vertebral body replacement device of claim 1, wherein at least one of the first and second bone contact surfaces define a plurality of spikes configured to engage adjacent bony structure.

6. The vertebral body replacement device of claim 1, wherein the first housing comprises a toothed interior surface and wherein the second housing comprises a toothed exterior surface that is complementary to the toothed interior surface of the first housing, wherein the engagement of the two toothed surfaces permits expansion of the vertebral body replacement device while preventing retraction of the vertebral body replacement device.

7. The vertebral body replacement device of claim 1, further comprising a retention member configured to engage an exterior surface of the first housing to restrict movement of the first housing with respect to the second housing.

8. The vertebral body replacement device of claim 7, wherein the retention member comprises a c-ring configured to at least partially wrap around the first housing and engage at least a portion of the external surface of the first housing.

9. A vertebral body replacement device comprising:
a first sleeve having an interior surface comprising a toothed surface;
and second sleeve having an exterior surface complimentary to the toothed surface;
wherein the first and second sleeves are configured to move longitudinally with respect to one another from the retracted position to the expanded position, and wherein the first and second sleeve define an interior cavity.

10. The vertebral body replacement device of claim 9, wherein the toothed surface comprises a plurality of teeth, wherein each tooth comprises a cam surface and a flat surface.

11. The vertebral body replacement device of claim 10, wherein when the vertebral body replacement device is moved from the retracted position to the expanded position, the cam surface of at least one of the teeth slides thereon a portion of the exterior surface of the second sleeve, permitting the first sleeve to slide in relation to the second sleeve, and when the vertebral body replacement device is moved from the expanded position to the retracted position, the flat surface of at least one of the teeth engages a portion of the exterior surface of the second sleeve, substantially preventing such movement.

12. The vertebral body replacement device of claim 11, wherein the exterior surface of the second sleeve is complimentarily toothed, and wherein the toothed surfaces are complimentarily opposite, meaning that in the expanding direction, the cam surface of a tooth on the second sleeve mates with the cam surface of a tooth on the first sleeve and, in the retracting direction, the flat surface of a tooth on the second sleeve engages the flat surface of a tooth on the first sleeve, permitting the two sleeves to move in the expanding direction in a ratcheting manner and preventing the two sleeves from moving in the retracting direction.

13. The vertebral body replacement device of claim 11, wherein the first sleeve comprises a toothed interior surface and the second sleeve comprises one or more complimentary recesses, where the recess has a flat interior edge that compliments the flat surface of at least one of the teeth of the first surface.

14. The vertebral body replacement device of claim 9, further comprising an upper bone contact member hingedly attached to a top portion of the second sleeve and a lower bone contact member hingedly attached to a lower portion of the first sleeve, permitting angulation of the bone contact members to compliment adjacent vertebral bodies.

15. The vertebral body replacement device of claim 14, wherein the first and second sleeves nest within one another and define a pillar window and the VBR device further comprises a pillar configured to fit within the interior cavity to be placed into the interior cavity via the pillar window, wherein a top portion of the pillar is angled and engages a portion of the upper bone contact member, and wherein and a bottom portion of the pillar is angled and engages a portion of the lower bone contact member, placing the upper and lower bone contact members at a desired angle for lordosis.

16. The vertebral body replacement device of claim 15, wherein the pillar, when positioned therein the interior cavity, substantially prevents the vertebral body replacement device from moving from the expanded position to the retracted position.

17. A method for replacing a vertebral body comprising the steps of:
accessing the desired motion segment;
removing the desired vertebral body;
positioning an expandable VBR device in place of the removed vertebral body;
expanding the expandable VBR device; and
fixing the expandable VBR device in the expanded position;
wherein the VBR device comprises:
a first housing defining an interior cavity;
a second housing nesting within at least a portion of the interior cavity of the first housing, the second housing moveable along a longitudinal direction to increase a height of the vertebral body replacement device, wherein the first housing can move in an expanding direction with respect to the second housing;
a first bone contact member having a first bone contact surface: and
a second bone contact member having a second bone contact surface.

18. The method of claim 17, wherein the second housing and first housing define a graft cavity for the placement of bone graft material and the method further comprises the step of packing the graft cavity with bone growth promoting materials.

19. The method of claim 17, wherein the first bone contact member is pivotally connected to a portion of a top portion of the first housing enabling the first bone contact member to angulate in either an anterior or posterior direction, and the second bone contact member is pivotally connected to a portion of a lower portion of the second housing enabling the second bone contact member to angulate in either an anterior or posterior direction.
